(19) United States
(12) Patent Application Publication
(10) Pub. No.: US 2022/0261495 A1
(43) Pub. Date: Aug. 18, 2022

(54) TERMINAL INFORMATION TRANSMISSION METHOD, AND RECORDING MEDIUM

(71) Applicant: NEC Corporation, Tokyo (JP)

(72) Inventor: Suguru KUSHIDA, Tokyo (JP)

(73) Assignee: NEC Corporation, Tokyo (JP)

(21) Appl. No.: 17/587,107

(22) Filed: Jan. 28, 2022

(30) Foreign Application Priority Data
Feb. 18, 2021 (JP) 2021-024371

(51) Int. Cl. G06F 21/62; G06F 21/32; A61B 3/11; G06V 40/18

(52) U.S. Cl. CPC G06F 21/6245; G06F 21/32; G06F 2221/2145; G06V 40/193; A61B 3/112

(57) ABSTRACT

A terminal information transmission device includes a person determination means configured to read biometric information of a subject person and determine whether the subject person is a subject person in question, an iris death determination means configured to execute a death determination by iris authentication when the subject person is determined to be the subject person in question by the person determination means, and a data transmission means configured to, when the iris death determination means determines that the subject person is dead, transmit belongings data stored in a subject person terminal of the subject person to a family member terminal of the subject person.

This application is based upon and claims the benefit of priority from Japanese Patent Application No. 2021-024371, filed on Feb. 18, 2021, the disclosure of which is incorporated herein in its entirety by reference.

### TECHNICAL FIELD

The present disclosure relates to a terminal information transmission device that causes stored data of a terminal owned by a subject person to be transmitted to a family member.

### BACKGROUND ART

After a family member has passed away, management of digital belongings such as digital data stored in a terminal or the like possessed by the deceased person is a work that takes time and effort for the family member left behind. With an aging society and an increase in the popularization rate of smart devices, demand for digital belongings management is expected to increase.

Accordingly, there is known a system that distributes information held by a deceased person when the deceased person passes away. Patent Literature 1 Japanese Patent Application Laid-open Publication 1 No. 2004-046294 discloses a deceased person information management device including a distribution means that distributes stored deceased person information when receiving a distribution request of the deceased person information. Patent Literature 2 Japanese Patent Application Laid-open Publication 2 No. 2012-168664 discloses a system in which a deceased person himself or herself records information regarding the deceased person himself or herself, relatives, and acquaintances while he or she is alive, and transmits the information recorded by the deceased person himself or herself to a limited range desired by the deceased person himself or herself using his or her death as a trigger. Patent Literature 3 Japanese Patent Application Laid-open Publication 3 No. 2016-081502 discloses an augmented reality system capable of obtaining information regarding a deceased person together with real information in a memorial facility.

As a method for diagnosing death of a deceased person, Patent Literature 4 Japanese Patent Application Laid-open Publication 4 No. 2020-156936 discloses a remote diagnosis method including notifying a doctor in charge of a patient of biological reaction stop information, and receiving information for death diagnosis obtained using a communication-type penlight camera by a caregiver of the patient who has been instructed by the doctor in charge who has received the notification.

### SUMMARY

An example of objects of the present disclosure is to provide a terminal information transmission device, a terminal information transmission method, and a non-transitory program storage medium capable of starting management of digital belongings of a deceased person without taking time and effort even when the digital belongings are not organized.

A terminal information transmission device according to one aspect of the present disclosure includes a memory; and at least one processor coupled to the memory, the at least one processor performing operations to:

read biometric information of a subject person;

determine whether the subject person is the subject person in question;

execute a death determination by iris authentication when the subject person is determined to be the subject person in question; and

transmit belongings data stored in a subject person terminal of the subject person to a family member terminal of the subject person when that the subject person is determined to be dead.

A terminal information transmission method according to one aspect of the present disclosure includes, by a computer, reading biometric information of a subject person and determining whether the subject person is a subject person in question, executing a death determination by iris authentication when the subject person is determined to be the subject person in question, and transmitting data stored in a subject person terminal of the subject person to a family member of the subject person when death of the subject is determined.

A non-transitory program storage medium according to one aspect of the present disclosure stores a program for causing a computer to execute reading biometric information of a subject person and determining whether the subject person is a subject person in question, executing a death determination by iris authentication when the subject person is determined to be the subject person in question, and transmitting data stored in a subject person terminal of the subject person to a family member of the subject person when death of the subject is determined.

### BRIEF DESCRIPTION OF THE DRAWINGS

Exemplary features and advantages of the present invention will become apparent from the following detailed description when taken with the accompanying drawings in which:

FIG. 1 is an exemplary block diagram illustrating a configuration of terminal information transmission according to a first example embodiment;

FIG. 2 is a diagram illustrating a hardware configuration example in which terminal information transmission according to the first example embodiment is achieved by a computer device and a peripheral device thereof;

FIG. 3 is an exemplary flowchart illustrating an operation of terminal information transmission according to the first example embodiment;

FIG. 4 is an exemplary block diagram illustrating a configuration of terminal information transmission according to a second example embodiment; and

FIG. 5 is an exemplary flowchart illustrating an operation of terminal information transmission according to the second example embodiment.

### EXAMPLE EMBODIMENT

Hereinafter, example embodiments will be described in detail with reference to the drawings.

### First Example Embodiment

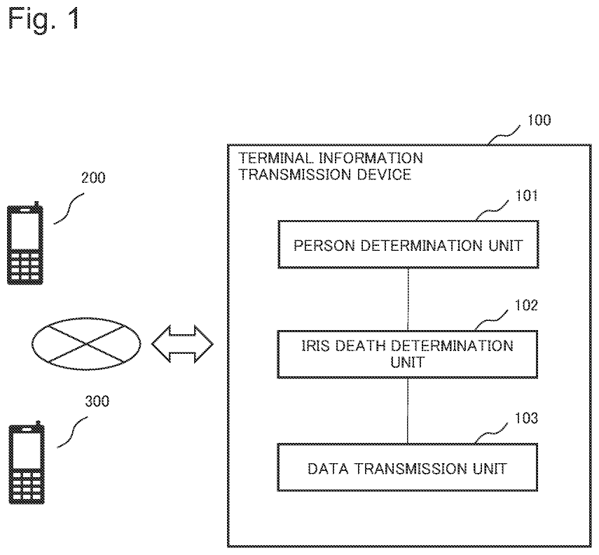

FIG. 1 is a block diagram illustrating a configuration of a terminal information transmission device 100 according to a first example embodiment. Referring to FIG. 1, the terminal information transmission device 100 includes a person determination unit 101, an iris death determination unit 102, and a data transmission unit 103. Next, a configuration of the terminal information transmission device 100 according to the first example embodiment will be described in detail.

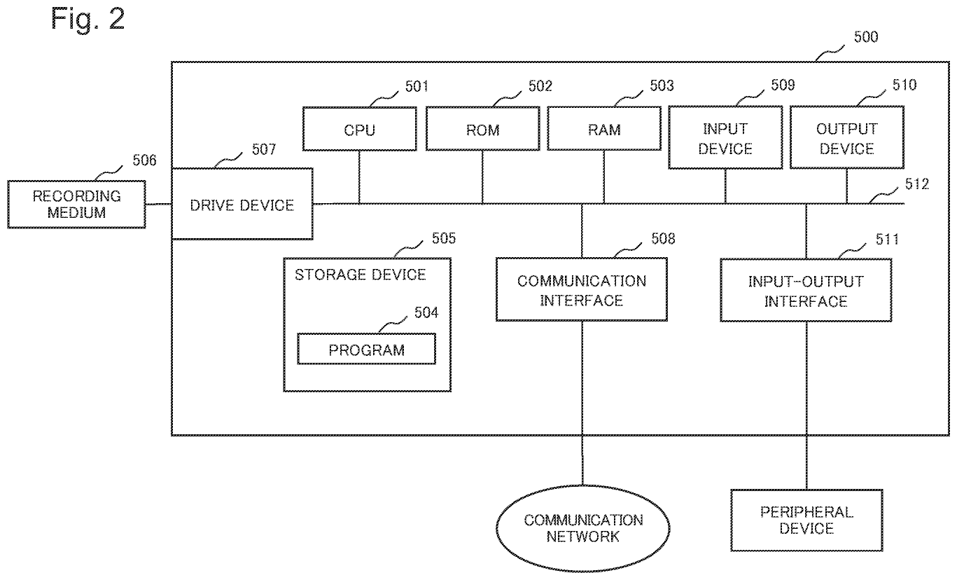

FIG. 2 is a diagram illustrating an example of a hardware configuration in which the terminal information transmission device 100 according to the first example embodiment of the present disclosure is achieved by a computer device 500 including a processor. As illustrated in FIG. 2, the terminal information transmission device 100 includes a central processing unit (CPU) 501, a memory including a read only memory (ROM) 502, a random access memory (RAM) 503, and the like, a storage device 505 such as a hard disk that stores a program 504, a communication interface (I/F) 508 for network connection, and an input-output interface 511 that inputs and outputs data. In the first example embodiment, digital data such as digital belongings stored in the terminal information transmission device 100 is transmitted from a subject person terminal 200 of a subject person to a family member terminal 300 via the input-output interface 511. The terminal information transmission device 100 is connected to an input device 509 and an output device 510 via a bus 512.

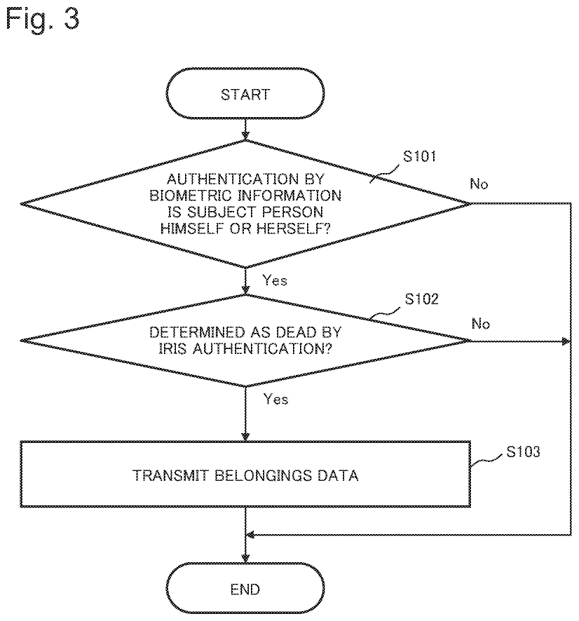

The CPU 501 operates an operating system to control the entire terminal information transmission device 100 according to the first example embodiment of the present invention. The CPU 501 reads a program and data from a recording medium 506 mounted on, for example, the drive device 507 or the like to the memory. The CPU 501 functions as a part of the person determination unit 101, the iris death determination unit 102, and the data transmission unit 103 in the first example embodiment, and executes processing or a command in a flowchart illustrated in FIG. 3 to be described later on the basis of a program.

The recording medium 506 is, for example, an optical disk, a flexible disk, a magnetic optical disk, an external hard disk, a semiconductor memory, or the like. A part of the recording medium of the storage device is a non-volatile storage device, and records a program therein. The program may be downloaded from an external computer (not illustrated) connected to a communication network.

The input device 509 is achieved by, for example, a mouse, a keyboard, a built-in key button, and the like, and is used for an input operation. The input device 509 is not limited to a mouse, a keyboard, and a built-in key button, and may be, for example, a touch panel. The output device 510 is achieved by, for example, a display, and is used to check an output.

As described above, the first example embodiment illustrated in FIG. 1 is achieved by the computer hardware illustrated in FIG. 2. However, means that achieve the units included in the terminal information transmission device 100 in FIG. 1 are not limited to the above-described configuration. Further, the terminal information transmission device 100 may be achieved by one physically coupled device, or may be achieved by a plurality of devices in which two or more physically separated devices are connected in a wired or wireless manner. For example, the input device 509 and the output device 510 may be connected to the computer device 500 via a network.

In FIG. 1, the person determination unit 101 is a unit that reads biometric information of a subject person and determines whether the subject person is a subject person in question. The subject person is a target who is a target of death determination. The biometric information is biometric information necessary for determining that the subject person is the subject person in question, and examples thereof include a fingerprint, a vein, a face, an iris, an ear shape, and the like, and a biometric authentication function that functions to release a lock of the subject person terminal 200 such as a personal computer (PC) or a smartphone owned by the subject person may be used. A contact address of a family member of the subject person is registered as a “contact address of a family member” in the subject person terminal 200, and data communication with the subject person terminal 200 is possible via the Internet, Air Drop™, or the like. When the biometric information of the subject person is read and the subject person is determined to be the subject person in question, the person determination unit 101 outputs a determination result that the subject person is the subject person in question to the iris death determination unit 102. When the biometric information of the subject person is read and the subject person is determined not to be the subject person in question, the person determination unit 101 outputs a determination result that the subject person is not the subject person in question to the iris death determination unit 102.

The iris death determination unit 102 is a unit that executes a death determination by iris authentication when the subject person is determined to be the subject person in question by the person determination unit 101. For example, when the determination result that the subject person is the subject person in question is input from the person determination unit 101, the iris death determination unit 102 performs a death determination by detecting the state of an iris such as presence or absence of pupil enlargement and presence or absence of light reflection. When it is determined that the subject person is dead from the state of the iris, the iris death determination unit 102 outputs a determination result indicating that the subject person is dead to the data transmission unit 103. When it is determined that the subject person is not dead from the state of the iris, the iris death determination unit 102 outputs a determination result indicating that the subject person is not dead to the data transmission unit 103.

The data transmission unit 103 is a unit that performs control to transmit belongings data stored in the subject person terminal 200 to the family member terminal 300 of the subject person when the iris death determination unit 102 determines that the subject person is dead. For example, when the determination result that the subject person has died is input by the iris death determination unit 102, the data transmission unit 103 controls the subject person terminal 200 to transmit the belongings data such as picture data to the family member terminal 300 by Air Drop™, a Web mail through the Internet, or the like. Specifically, the data transmission unit 103 transmits a picture and video data stored in the subject person terminal 200 to a destination registered as a contact address of the family member from an address book.

An operation of the terminal information transmission device 100 configured as described above will be described with reference to the flowchart of FIG. 3.

FIG. 3 is a flowchart illustrating an outline of an operation of the terminal information transmission device 100 according to the first example embodiment. Note that the processing according to this flowchart may be executed on the basis of program control by the processor described above.

As illustrated in FIG. 3, first, the person determination unit 101 reads biometric information of the subject person and determines whether the subject person is the subject person in question (step S101). When it is determined that the subject person is the subject person in question (step S101; Yes), the person determination unit 101 outputs a result of determination that the subject person is the subject person in question to the iris death determination unit 102. When it is determined that the subject person is not the subject person in question (step S101; No), the person determination unit 101 ends the terminal information transmission operation.

Next, when it is determined that the subject person is the subject person in question, the iris death determination unit 102 executes the death determination by the state of the iris (step S102). When it is determined that the subject person has died (step S102; Yes), the iris death determination unit 102 outputs a result of determination that the subject person is dead to the data transmission unit 103.

Next, when the result of determination that the subject person is dead is input to the data transmission unit 103, the data transmission unit 103 automatically transmits the data stored in the subject person terminal 200 to the family member of the subject person.

Thus, the terminal information transmission device 100 ends the operation of the terminal information transmission.

Next, effects of the first example embodiment of the present disclosure will be described. The terminal information transmission device 100 according to the present example embodiment described above can start management of digital belongings of the deceased person without taking time and effort even when the digital belongings are not organized. The reason is that when the subject person is determined to be the subject person in question by the person determination unit 101 and the subject person is determined to have died by the iris death determination unit 102, the data stored in the subject person terminal 200 is automatically transmitted to the family member terminal 300. Conventionally, when an analysis company is requested to analyze a password set in a terminal of a deceased person, it is necessary to submit a document proving the death such as a death notification and the procedure is complicated, but the terminal information transmission device 100 can save time and effort.

### Second Example Embodiment

Next, a second example embodiment of the present disclosure will be described in detail with reference to the drawings. Hereinafter, description of contents overlapping with the above description will be omitted to the extent that the description of the present example embodiment is not unclear.

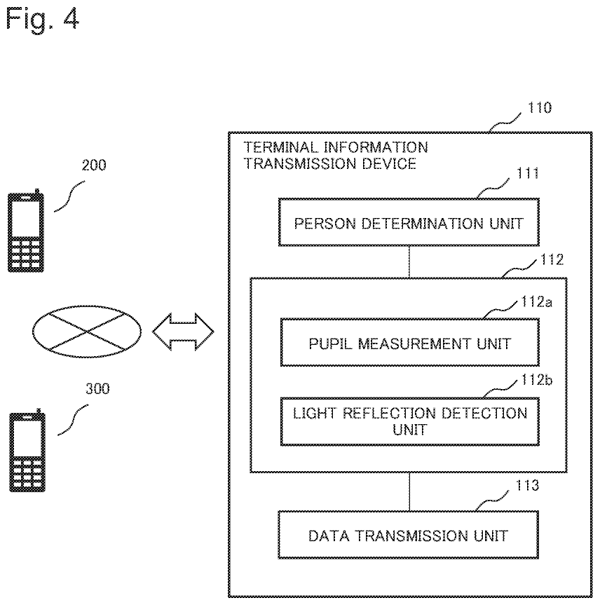

FIG. 4 is a block diagram illustrating a configuration of a terminal information transmission device 110 according to the second example embodiment of the present disclosure. With reference to FIG. 4, the terminal information transmission device 110 according to the second example embodiment will be described focusing on a part different from the terminal information transmission device 100 according to the first example embodiment. Operations and functions of the person determination unit 111 of the terminal information transmission device 110 according to the second example embodiment are similar to those of the person determination unit 101 in the first example embodiment, and thus detailed description thereof will be omitted. In the second example embodiment of the present disclosure, belongings data to be shared with the family member after death of a subject person is stored in the subject person terminal 200 in advance in a manner of being distinguished from other data before while the subject person is alive.

The terminal information transmission device 110 according to the present example embodiment includes a pupil measurement unit 112a and a light reflection detection unit 112b in the iris death determination unit 112. The pupil measurement unit 112a measures a size of a pupil, and determines that the pupil is open when the size of the pupil of the subject person is larger than a threshold (for example, equal to or more than 5 mm). When the pupil measurement unit 112a determines that the pupil is opened, the light reflection detection unit 112b determines death based on presence or absence of disappearance of light reflection of the subject person using the iris authentication. The iris death determination unit 112 irradiates the subject person with light, determines that the subject person has died (including brain death) when the light reflection is not detected, and outputs a result to the data transmission unit 113. In this case, the light reflection detection unit 112b does not release the screen lock of the subject person terminal 200 in order to prevent a third party from abusing the data on the terminal. On the other hand, the light reflection detection unit 112b irradiates the pupil of the subject person with light, and in a case where light reflection is detected, the light reflection detection unit 112b notifies the display unit of the subject person terminal 200 that there is a suspicion of cranial neuropathy, and releases the screen lock of the subject person terminal 200.

The data transmission unit 113 according to the second example embodiment includes, in addition to means of the data transmission unit 103 according to the first example embodiment, a unit that detects a response by transmitting a command to display a confirmation screen as to whether to acquire the belongings data to the subject person terminal 200 before transmitting the belongings data of the subject person to the family member terminal 300 of the subject person, when the iris death determination unit 112 determines that the subject person is dead. When a result of determination that the subject person is dead is input from the iris death determination unit 112 and the data transmission unit 113 transmits the above-described command, the light reflection detection unit 112b first releases the screen lock of the subject person terminal 200 and causes a screen notifying that the death determination is made to be displayed on the display unit (not illustrated) of the subject person terminal 200. At the same time, the iris death determination unit 112 causes a confirmation screen as to whether to acquire the belongings data of the subject person to be displayed on the display unit of the subject person terminal 200. Next, the data transmission unit 113 detects presence or absence of a response to an operation on the confirmation screen. That is, when it is detected that there is an input to acquire the belongings data on the confirmation screen, the data transmission unit 113 instructs the subject person terminal 200 to automatically transmit the belongings data of pictures, mails, a telephone book, and the like stored in the subject person terminal 200 from the subject person terminal 200 to the family member terminal 300 of the subject person. On the other hand, when it is detected that there is an input not to acquire the belongings data on the confirmation screen, the data transmission unit 113 does not transmit the belongings data of the pictures, mails, telephone book, and the like stored in the subject person terminal 200 to the family member terminal 300 of the subject person.

The data transmission unit 113 causes a confirmation screen to be displayed as to whether to perform a cancellation procedure of an app or the like installed in the subject person terminal 200. When it is detected that information indicating cancellation of the app is input on the confirmation screen, the data transmission unit 113 notifies an app operating company or the like to perform a procedure necessary for cancellation. On the other hand, when it is detected that information indicating that the app is not canceled is input on the confirmation screen, the data transmission unit 113 does not notify the app operating company or the like to perform a procedure necessary for cancellation. Here, the installed app includes, for example, a social networking service (SNS) on an Internet service, and an app of a securities company or a network bank. The SNS is provided with an ID and a password to the subject person while the subject person is alive, and functions such as browsing and posting on the subject person terminal 200 can be used when the ID and the password are input. The apps of the securities company and the Internet bank allow transactions when the subject person opens these accounts while the subject person is alive and enters an ID and a password in the app installed on the subject person terminal 200.

The functions of the components according to each example embodiment of the present disclosure described above can be achieved not only by hardware but also by a computer device and firmware based on program control, similarly to the computer device illustrated in FIG. 2. In the second example embodiment, a response to acquisition of belongings data by a family member of a subject person and necessity of a cancellation procedure of an app is input to the terminal information transmission device 110 via the input-output interface 511.

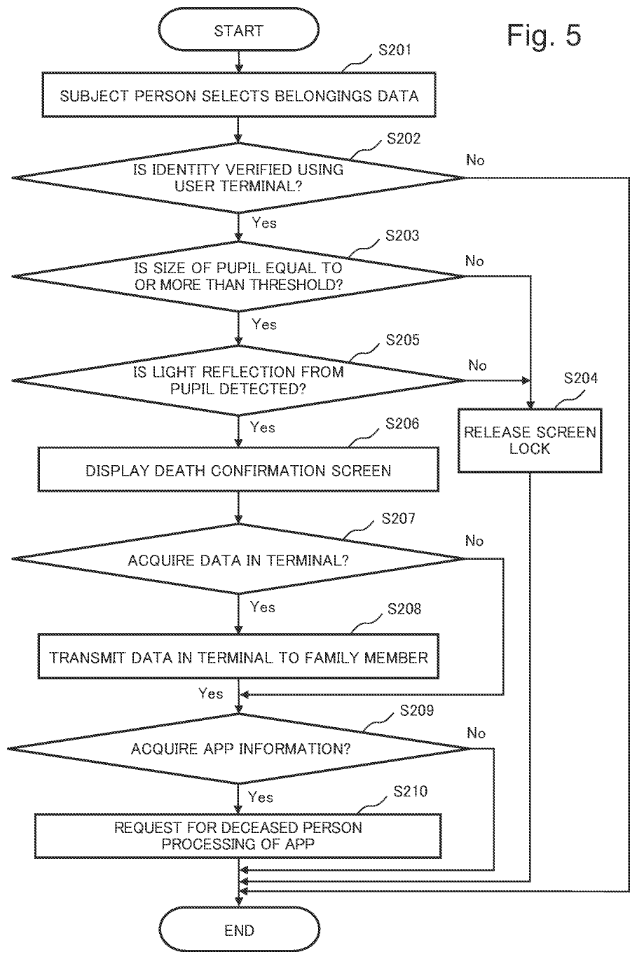

Next, the operation of the terminal information transmission device 110 will be described with reference to a flowchart illustrated in FIG. 5. Note that the processing according to the flowchart may also be executed on the basis of the program control by the CPU described above.

FIG. 5 is a flowchart illustrating an outline of an operation of terminal information transmission in the terminal information transmission device 110 according to the second example embodiment.

As illustrated in FIG. 5, first, the subject person selects data to be shared with the family member as belongings data among the data stored in the subject person terminal 200 while the subject person is alive (step S201).

Next, when a situation to determine whether the subject person has died occurs, the person determination unit 111 reads the biometric information of the subject person and determines whether the subject person is the subject person in question (step S202). When it is determined that the subject person is the subject person in question (step S202; Yes), the person determination unit 111 outputs a result of determination that the subject person is the subject person in question to the iris death determination unit 112. When it is determined that the subject person is not the subject person in question (step S202; No), the person determination unit 101 ends the flow.

Next, the pupil measurement unit 112a of the iris death determination unit 102 measures the size of the pupil, and determines whether the subject person has died according to whether the size of the pupil of the subject person is larger than a threshold (for example, equal to or more than 5 mm) (step S203). When the size of the pupil of the subject person is larger than the threshold (step S203; Yes), the pupil measurement unit 112a determines that the pupil is opened, and outputs a result thereof to the light reflection detection unit 112b. When the size of the pupil of the subject person is less than the threshold (step S203; No), the pupil measurement unit 112a determines that the pupil is not opened, and releases the screen lock of the subject person terminal 200 (step S204) and ends the flow.

Next, the light reflection detection unit 112b of the iris death determination unit 102 determines whether the subject person is dead according to presence or absence of light reflection disappearance of the subject person (step S205).

When the light reflection detection unit 112b irradiates the subject person with light and no light reflection is detected (step S205; Yes), it is determined that the subject person has died, and a result thereof is output to the data transmission unit 113. On the other hand, when the light reflection detection unit 112b detects light reflection after irradiating the subject person with light (step S205; No), it is determined that the subject person has not died (it is determined that there is a suspicion of brain death), and a suspicion of cranial neuropathy is notified on the display unit of the subject person terminal 200, the screen lock of the subject person terminal 200 is released (step S204), and the flow is terminated.

Next, when the result of determination that the subject person is dead is input from the light reflection detection unit 112b, the data transmission unit 113 causes a confirmation screen for confirming whether to acquire the belongings data to be displayed on the screen of the subject person terminal 200 (step S206).

When the data transmission unit 113 detects that there is an input to acquire the belongings data on the confirmation screen (step S207; Yes), it is automatically caused to transmit the belongings data of the picture, text, and the like in the subject person terminal 200 to the family member terminal 300 of the subject person (step S208). On the other hand, when the data transmission unit 113 detects that there is an input to acquire the belongings data on the confirmation screen (step S207; No), the flow proceeds to the next step without automatically transmitting the belongings data of the picture, text, and the like in the subject person terminal 200 to the family member terminal 300 of the subject person.

Next, the data transmission unit 113 causes a confirmation screen to be displayed as to whether to perform a cancellation procedure of an app or the like installed in the subject person terminal 200. The confirmation screen for confirming whether to proceed with app cancellation is displayed on the screen of the subject person terminal 200 (step S209).

When it is detected that the cancellation procedure of the app is performed on the confirmation screen (step S209; Yes), the data transmission unit 113 notifies the app operating company or the like of performing a procedure necessary for cancellation (step S210). On the other hand, when it is detected that there is an input not to perform cancellation procedure of the app on the confirmation screen (step S209; No), the data transmission unit 113 ends the flow without giving a notification of performing a cancellation procedure of the app.

Thus, the terminal information transmission device 110 ends the operation of the terminal information transmission.

The confirmation as to whether the subject person is the subject person in question executed by the person determination unit 111 (step S202) executed by the terminal information transmission device 110 is similar to that in the first example embodiment.

Next, effects of the second example embodiment of the present disclosure will be described. In the terminal information transmission device 110 according to the second example embodiment described above, the pupil measurement unit 112a includes a pupil measurement unit that measures a size of a pupil, and determines that the subject person is dead when the size of the pupil of the subject person is larger than a threshold (for example, equal to or more than 5 mm) and the light reflection detection unit 112b determines that the light reflection has disappeared. In this case, even if the size of the pupil is larger than the threshold, if the light reflection has not disappeared, a notification that there is a cranial nerve suspicion is given, and it is not determined that the subject person is dead. Therefore, in the second example embodiment, since the death confirmation is performed in a plurality of steps, it is possible to perform accurate death determination.

In the terminal information transmission device 110 according to the second example embodiment, data that may be shared with the family member after the subject person dies is selected in S201 in advance, and the selected data is stored in the subject person terminal 200, so that a choice of data to be left to the family member can be made.

While the invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims.

For example, although a plurality of operations is described in order in the form of a flowchart, the order of description does not limit the order of executing the plurality of operations. Thus, when each example embodiment is implemented, the order of the plurality of operations can be changed within a range that does not interfere in content. In the second example embodiment, after the confirmation screen as to whether to acquire the data stored in the subject person terminal 200 is displayed in S207, the data in the subject person terminal 200 is transmitted to the family member terminal 300, and then the confirmation screen as to necessity of cancellation of the app downloaded to the subject person terminal 200 is displayed in S209. However, acquisition of the data in the subject person terminal 200 and necessity of the app cancellation may be simultaneously displayed on the screen. After the confirmation screen as to necessity of cancellation of the app is displayed first, the confirmation screen as to whether the data in the subject person terminal 200 has been acquired may be displayed.

In the second example embodiment, a notification that there is a suspicion of cranial neuropathy in S204, the confirmation screen for confirming whether to acquire the belongings data in S206, and the confirmation screen for confirming whether to perform the cancellation procedure of the app or the like in S206 are displayed on the screen of the subject person terminal 200. However, these notifications and screens are not limited to display on the screen of the subject person terminal 200. These confirmation screens may be displayed using, for example, a display screen of the family member terminal 300 of the subject person or other display devices.

In the second example embodiment, the belongings data to be shared with the family member after the death of the subject person is stored in the subject person terminal 200 in advance in a manner of being distinguished from other data. However, the belongings data to be shared with the family member may be stored not in the subject person terminal 200 but in the storage device 505 of the terminal information transmission device 110. In this case, the terminal information transmission device 110 itself transmits the belongings data to the family member terminal 300.

In the second example embodiment, instead of the person determination unit 111, the pupil measurement unit 112a, and the light reflection detection unit 112b in the terminal information transmission device 110, a biometric authentication function of authenticating whether a subject person is the subject person in question and an iris authentication function capable of measuring presence or absence of pupil enlargement, presence or absence of light reflection, and the like, which are included in the subject person terminal 200 itself, may be used. In this case, the determination of the subject person in question by the person determination unit 111 and the death determination by the pupil measurement unit 112a and the light reflection detection unit 112b can be performed with one subject person terminal 200. Thus, by simply holding the subject person terminal 200 over the subject person, the belongings data is transmitted to the family member terminal 300 of the subject person, and the family member can start management of the belongings data. Even in this case, the data transmission unit 113 may be in a device different from the subject person terminal 200, and control may be performed to transmit the belongings data in the subject person terminal 200 to the family member terminal 300. The data transmission unit 113 may be in the subject person terminal 200, and the subject person terminal 200 itself may transmit the belongings data in the subject person terminal 200 to the family member terminal 300.

The invention described in Patent Literatures 1 to 3 requires an ID and a password for sharing digital belongings of a deceased person in order to share data of the deceased person with family members. In a case where the deceased person died suddenly or in a case where digital belongings are not organized, such as a case where a family member does not know a password, it is necessary to request a business operator or the like to analyze the password. The diagnosis method described in Patent Literature 4 requires a death diagnosis by a doctor, and it takes time and effort such that a death diagnosis result by a doctor is required when a family member starts management of digital belongings of a deceased person.

An example of an effect of the present invention is that it is possible to start management of digital belongings of a deceased person without taking time and effort even when digital belongings are not organized.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

1. A terminal information transmission device comprising:
a memory; and
at least one processor coupled to the memory,
the at least one processor performing operations to:
read biometric information of a subject person;
determine whether the subject person is the subject person in question;
execute a death determination by iris authentication when the subject person is determined to be the subject person in question; and
transmit belongings data stored in a subject person terminal of the subject person to a family member terminal of the subject person when that the subject person is determined to be dead.

2. The terminal information transmission device according to claim 1, wherein the at least one processor is further configured to
measure a size of a pupil; and
determine that the subject person is dead when the size of the pupil of the subject person is larger than a threshold.

3. The terminal information transmission device according to claim 2, wherein the at least one processor is further configured to
detect presence or absence of reflected light from a pupil,
irradiate the pupil of the subject person with light when the size of the pupil of the subject person is larger than the threshold; and
determine that the subject person is dead when reflected light from the pupil is detected.

4. The terminal information transmission device according to claim 1, wherein he at least one processor is further configured to
cause only data selected in advance by the subject person to be transmitted to a family member of the subject person.

5. The terminal information transmission device according to claim 1, wherein the at least one processor is further configured to
cause a confirmation screen to be displayed as to whether to transmit the belongings data to the family member terminal before transmitting the belongings data to the family member terminal of the subject person when determined that the subject person is dead; and
transmit the belongings data to the family member terminal of the subject person when a response to transmit the belongings data to the family member terminal is detected on the confirmation screen.

6. The terminal information transmission device according to claim 1, wherein the at least one processor is further configured to
determine whether the subject person is the subject person in question by using an authentication function of a terminal of the subject person.

7. The terminal information transmission device according to claim 1, wherein the at least one processor is further configured to
determine whether the subject person is dead using an iris authentication function of a terminal of the subject person.

8. A terminal information transmission method comprising, by a computer:
reading biometric information of a subject person and determining whether the subject person is a subject person in question;
executing a death determination by iris authentication when the subject person is determined to be the subject person in question; and
transmitting data stored in a subject person terminal of the subject person to a family member of the subject person when death of the subject is determined.

9. A non-transitory program storage medium storing a program for causing a computer to execute:
reading biometric information of a subject person and determining whether the subject person is a subject person in question;
executing a death determination by iris authentication when the subject person is determined to be the subject person in question; and
transmitting data stored in a subject person terminal of the subject person to a family member of the subject person when death of the subject is determined.
